(19) United States
(12) Patent Application Publication
(10) Pub. No.: US 2026/0047864 A1
(43) Pub. Date: Feb. 19, 2026

(54) A SHARPS DISLODGER

(71) Applicant: Keng Kiong TAY, Kuching (MY)

(72) Inventor: Keng Kiong TAY, Kuching (MY)

(21) Appl. No.: 19/100,472

(22) PCT Filed: Feb. 22, 2023
(86) PCT No.: PCT/MY2023/050011

§ 371 Date: Jan. 31, 2025

(30) Foreign Application Priority Data
Aug. 1, 2022 (MY) PI2022004108

(51) Int. Cl. A61B 17/3217; A61B 17/3213; A61B 90/00

(52) U.S. Cl. CPC A61B 17/3217; A61B 17/3213; A61B 90/08; A61B 2090/0801

(57) ABSTRACT

The present invention relates a sharps dislodger (100), comprising: a wall (105) having an opening (102) for receiving a blade of a medical sharp; characterized by: at least a pair of static spokes (103) disposed below the opening having a slit (104) at one end; wherein the slit is slanted at 30°angles for receiving the blade; whereby the pair of static spokes (103) breaks the blade with handle as the blade moves upright to 90° angles and to remove from the handle.

### TECHNICAL FIELD

The present invention generally relates to a sharp dislodge and more particularly to a manual blade removal and disposal in a safe manner.

### BACKGROUND ART

A medical sharp bin comprising a sharps inlet for needle removal and blade removal is widely known. The conventional blade removal comprises a complicated mechanism of lifting and pulling out the handle to dislodge the scalpel blade.

There have been a number of solutions that provide a manual dislodger for medical sharps and two of them are discussed below:

EP0569233A1 relates to a sharps handling apparatus that includes a blade removing device for removing a scalpel blade from a scalpel handle of the conventional type having a tang that retains the blade. The blade-removing device is located in a wall which in the described embodiment is provided in the lid of a sharps container. There is an opening in the wall and a ramp that is inclined at an acute angle to the wall adjacent to the opening. A slot is formed in the ramp and extends down the incline and there is a detent on the ramp. The arrangement is such that when the scalpel blade is pressed into the opening in the wall, the blade retaining tang can ride through the slot while the trailing end of the blade is obliged to ride along the operative underside of the ramp. This causes the blade to flex to a condition of disengagement with the tang. The blade is then engaged by the detent so that the scalpel handle can be separated from the blade. In the described embodiment, the blade then falls into the sharps container for safe disposal. The described embodiment also has the facility for removing hypodermic and other needles and for storing sutures in a safe and stick-free manner. However, the prior art relies on a complicated mechanism to disengage the used scalpel blade intact from the tang.

WO1996007363A1 discloses a scalpel blade remover that removes a blade from the tang of a scalpel blade handle. The scalpel is inserted in and withdrawn from, and the blade removed along the same substantially straight-line path. The blade remover has a rocker block that pivots about an axis transverse to the insertion path. When the scalpel is inserted into the blade remover, its tang pushes against a contact surface of the rocker block, causing the block to pivot. A pair of legs at the other end of the block applies a transverse force to the heel of the blade on either side of the tang, thereby lifting the heel of the blade off the tang. When the scalpel has been inserted far enough, a pawl drops behind the heel of the blade. The pawl strips the blade from the tang when the scalpel handle is removed. The blade remover is typically mounted on the lid of a sharps container. However, the prior art also relies on complicated mechanism to disengage the used scalpel blade intact from the tang.

Accordingly, it can be seen in the prior arts that there is a need to have an improved manual sharps inlet that safely removes the used scalpel blade using a simpler mechanism by breaking it.

### SUMMARY OF THE INVENTION

The following presents a simplified summary of the invention in order to provide a basic understanding of some aspects of the invention. This summary is not an extensive overview of the invention. Its sole purpose is to present some concepts of the invention in a simplified form as a prelude to the more detailed description that is presented later.

An objective of the present invention is to provide a safe manual sharps inlet that safely removes the used scalpel blade by breaking it.

Accordingly, these objectives may be achieved by following the teachings of the present invention. The present invention relates a sharps dislodger, comprising: a wall having an opening for receiving a blade of a medical sharp; characterized by: at least a pair of static spokes disposed below the opening having a slit at one end; wherein the slit is slanted at 30° angles for receiving the blade; whereby the pair of static spokes breaks the blade with handle as the blade moves upright to 90° angles and to remove from the handle.

The foregoing and other objects, features, aspects and advantages of the present invention will become better understood from a careful reading of a detailed description provided herein below with appropriate reference to the accompanying drawings.

### BRIEF DESCRIPTION OF THE ACCOMPANYING DRAWINGS

So that the manner in which the above recited features of the present invention can be understood in detail, a more particular description of the invention, briefly summarized above, may have been referred by embodiments, some of which are illustrated in the appended drawings. It is to be noted, however, that the appended drawing illustrates only typical embodiments of this invention and are therefore not to be considered limiting of its scope, for the invention may admit to other equally effective embodiments.

These and other features, benefits, and advantages of the present invention will become apparent by reference to the following text figure, with like reference numbers referring to like structures across the views, wherein:

FIG. 1 is a diagram illustrating a sharps dislodger in accordance to an embodiment of the present invention;

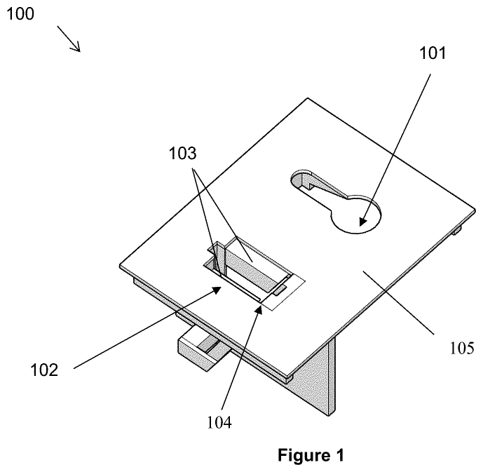

FIG. 2 is a diagram illustrating a scalpel blade removal mechanism using the sharps dislodge in accordance to an embodiment of the present invention; and

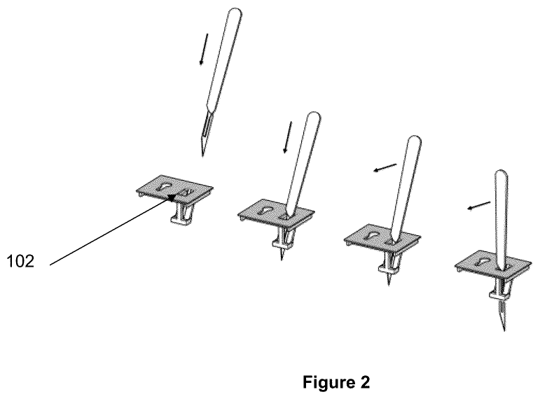

FIG. 3 is a diagram illustrating a manual blade remover mechanism in: (a) a pre-use position in 30° angle, (b) a manual breaking position in 90° angle, and (c) an end-use position in accordance to an embodiment of the present invention.

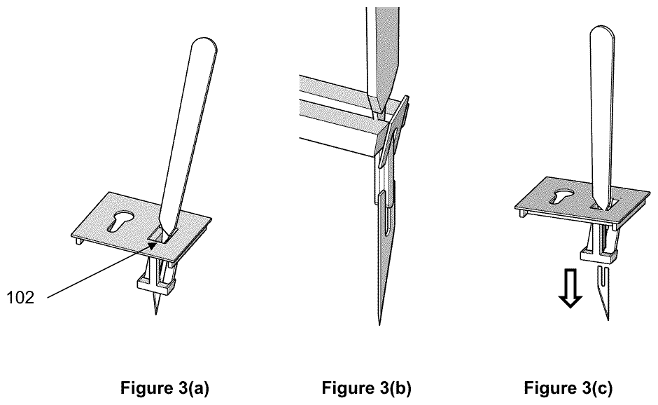

### DETAILED DESCRIPTION OF THE PREFERRED EMBODIMENTS

While the present invention is described herein by way of example using embodiments and illustrative drawings, those skilled in the art will recognize that the invention is not limited to the embodiments of drawing or drawings described, and are not intended to represent the scale of the various components. Further, some components that may form a part of the invention may not be illustrated in certain figures, for ease of illustration, and such omissions do not limit the embodiments outlined in any way. It should be understood that the drawings and detailed description thereto are not intended to limit the invention to the particular form disclosed, but on the contrary, the invention is to cover all modifications, equivalents, and alternatives falling within the scope of the present invention as defined by the appended claim. As used throughout this description, the word “may” is used in a permissive sense (i.e. meaning having the potential to), rather than the mandatory sense, (i.e. meaning must). Further, the words “a” or “an” mean “at least one” and the word “plurality” means “one or more” unless otherwise mentioned. Furthermore, the terminology and phraseology used herein is solely used for descriptive purposes and should not be construed as limiting in scope. Language such as “including,” “comprising,” “having,” “containing,” or “involving,” and variations thereof, is intended to be broad and encompass the subject matter listed thereafter, equivalents, and additional subject matter not recited, and is not intended to exclude other additives, components, integers or steps. Likewise, the term “comprising” is considered synonymous with the terms “including” or “containing” for applicable legal purposes. Any discussion of documents, acts, materials, devices, articles and the like is included in the specification solely for the purpose of providing a context for the present invention. It is not suggested or represented that any or all of these matters form part of the prior art base or were common general knowledge in the field relevant to the present invention.

In this disclosure, whenever a composition or an element or a group of elements is preceded with the transitional phrase “comprising”, it is understood that we also contemplate the same composition, element or group of elements with transitional phrases “consisting of”, “consisting”, “selected from the group of consisting of, “including”, or “is” preceding the recitation of the composition, element or group of elements and vice versa.

The present invention is described hereinafter by various embodiments with reference to the accompanying drawing, wherein reference numerals used in the accompanying drawing correspond to the like elements throughout the description. This invention may, however, be embodied in many different forms and should not be construed as limited to the embodiment set forth herein. Rather, the embodiment is provided so that this disclosure will be thorough and complete and will fully convey the scope of the invention to those skilled in the art. In the following detailed description, numeric values and ranges are provided for various aspects of the implementations described. These values and ranges are to be treated as examples only and are not intended to limit the scope of the claims. In addition, a number of materials are identified as suitable for various facets of the implementations. These materials are to be treated as exemplary and are not intended to limit the scope of the invention.

With reference to the drawings, the invention will now be described in more detail.

The present invention relates a sharps dislodger (100), comprising: a wall (105) having an opening (102) for receiving a blade of a medical sharp; characterized by: at least a pair of static spokes (103) disposed below the opening having a slit (104) at one end; wherein the slit is slanted at 30° angles for receiving the blade; whereby the pair of static spokes (103) breaks the blade with handle as the blade moves upright to 90° angles and to remove from the handle.

In accordance with an embodiment of the present invention, the sharps dislodger (100) mounted to a medical sharps bin.

In accordance with an embodiment of the present invention, the blade with handle is a scalpel blade.

In accordance with an embodiment of the present invention, the sharps dislodger further comprising an elongated opening (101) for needle removal.

Various modifications to these embodiments are apparent to those skilled in the art from the description and the accompanying drawings. The principles associated with the various embodiments described herein may be applied to other embodiments. Therefore, the description is not intended to be limited to the embodiments shown along with the accompanying drawings but is to be providing broadest scope of consistent with the principles and the novel and inventive features disclosed or suggested herein. Accordingly, the invention is anticipated to hold on to all other such alternatives, modifications, and variations that fall within the scope of the present invention and appended claim.

It is to be understood that any prior art publication referred to herein does not constitute an admission that the publication forms part of the common general knowledge in the art.

In the claims which follow and in the preceding description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

1. A sharps dislodger comprising: a wall having an opening for receiving a blade of a medical sharp;
characterized by:
at least a pair of static spokes disposed below the opening having a slit at one end; wherein the slit is slanted at a 30° angle for receiving the blade;
whereby the pair of static spokes breaks the blade with a handle as the blade moves upright to 90° angle and to remove from the handle.

2. The sharps dislodger as claimed in claim 1, wherein the sharps dislodger is mounted to a medical sharps bin.

3. The sharps dislodger as claimed in claim 1, wherein the blade with the handle is a scalpel blade.
